(19) United States
(12) Patent Application Publication
(10) Pub. No.: US 2010/0173010 A1
(43) Pub. Date: Jul. 8, 2010

(54) Polymetaphosphate based formulations for therapy of microcrystalline arthropathies

(75) Inventors: Roberto Marcolongo, Siena (IT); Manuela Catenaccio, Siena (IT); Daniela Chindamo, Siena (IT); Sauro Lorenzini, Siena (IT); Enrico Selvi, Siena (IT); Renzo Cini, Siena (IT); Gabriella Tamasi, Siena (IT); Michele Gregorkiewitz, Asciano (IT); Giovanni Cavallo, Roma (IT)

Correspondence Address: LUCAS & MERCANTI, LLP, 475 PARK AVENUE SOUTH, 15TH FLOOR, NEW YORK, NY, 10016, US

(73) Assignee: UNIVERSITÁ DEGLI STUDI DI SIENA, Siena (IT)

(21) Appl. No.: 10/583,605

(22) PCT Filed: Dec. 21, 2004
(86) PCT No.: PCT/IT04/00713

§ 371 Date: Nov. 30, 2009

(30) Foreign Application Priority Data
Dec. 22, 2003 (IT) RM2003A000590

(51) Int. Cl. A61K 33/42; A61P 19/02; C01B 25/30

(52) U.S. Cl. 424/605; 423/314

(57) ABSTRACT

Soluble pharmaceutical composition for the treatment of articular pathologies comprising an effective amount of a least one linear or cyclic polymetaphosphate or a soluble and pharmaceutically acceptable salt thereof, and appropriate diluents.

The present invention relates to polymetaphosphate-based composition for therapy of microcrystalline arthropathies.

### BACKGROUND ART

Microcrystalline arthropathies are a group of inflammatory-degenerative pathologies, characterized by the deposition of mineral substances in articular and periarticular structures in crystalline form. In particular, chondrocalcinosis is a disease characterized by microcrystalline deposits of calcium pyrophosphate dihydrate, Ca₂[O(PO₃)₂](2H₂O) (CPPD). In the course of chondrocalcinosis, synovitic episodes secondary to the release of CPPD crystals from tissue deposits in the synovial frequently occur. The identification of crystals in the synovial liquid of patients with gout-like arthritis was described in 1962 by McCarthy [McCarthy D J Jr, Kohn N N, Faires J s. The significance of calcium phosphate crystal in the synovial fluid of arthritis patients, the pseudogout syndrome. Clinical aspects. Ann Intern Med 56: 711-737 (1962)].

Another common microcrystalline arthropathy is caused by the deposit of hydroxyapatite crystals, Ca₅(PO₄)₃OH(HAP), at the articular and periarticular level. Usually, this pathology manifests itself in association with other arthropathies of a pre-eminently degenerative nature such as osteoarthrosis, calcific periarthritis, tendinitis and calcific bursitis. Although calcific deposits are often not associated to specific clinical specifications, they can assume particular relevance in conditions such as calcific periarthritis of the shoulder, in which it is believed that such calcifications are partly responsible for the inflammatory degenerative manifestations of periarticular structure [Dieppe P A, Crocker P, Huskisson E C, Willoughby A D. Apatite deposition disease: a new arthropathy. Lancet 1: 266-268 (1976)].

The mechanism that leads to the precipitation and deposition of CPPD or HAP crystals is not yet known, nor does it appear clear whether degenerative alterations of the cartilage are primitive or secondary to the deposition of the crystals. The likeliest hypothesis is that this deposition is due to a local metabolic alteration. In case of chondrocalcinosis, the pyrophosphate produced by the chondrocytes would be diffused in the fundamental substance according to an increased synthesis or to a tissue inability to hydrolyze the compound with pyrophosphatase enzymes, including alkaline phosphatase. Small deposits of pyrophosphate are often observed in the cartilage of elderly subjects, especially as a result of an increased synthesis and concentration of pyrophosphates, by “nucleoside triphosphate pyrophosphohydrolase (NTPPPH) enzymes [Ryan M L, McCarthy D J. Calcium Pyrophosphate Crystal Deposition Disease; Psedogout; Articular Chondrocalcinosis. In: Arthritis and Allied Conditions: A Textbook of Rheumatology (D. J. McCarthy & W. J. Koopman eds.), vol. 2 (12^{th} Ed.), Philadelphia, Pa., Lippincott Williams & Wilkins, pp. 1835-1855 (1993)]. In turn, pyrophosphates are an important source of inorganic phosphates, which have a fundamental role in bone mineralization. There is an equilibrium between pyrophosphates and phosphates: when the former prevail, they precipitate in crystalline form; when phosphates prevail, there a greater solubilization and reduction of pyrophosphate crystals [Anderson H C. Mechanisms of pathologic calcification. Rheum Dis Clin Am 14: 303-319 (1988); Rosen F, McCabe G, Quach J, Solan J, Terkeltaub R, Seegmiller J E, Lotz M. Differential effects of aging on human chondrocyte responses to transforming growth factor: increased pyrophosphate production and decreased cell proliferation. Arthritis Rheum 40: 1275-1281 (1997)].

CPPD crystals have elongated rhomboidal shape, although at times they are highlighted in the shape of long or short rods and small squares, whereas HAP crystals are smaller and have needle or rod shape. Currently, it is believed that acute pseudogout attacks are due to the release into the articular cavity (synovial liquid) of CPPD crystals, which are coated (opsonized) with proteins (especially IgG) and then recognized and phagocytosed by polymorphonuclear neutrophils (PMN). During phagocytosis and the subsequent cell destruction, lysosomal enzymes, reactive oxygen species (ROS), leucotriens, are released which act as chemical mediators of the inflammation, with consequent acute arthritis or pseudogout [Burt H M, Jackson J K. Enhancement of crystal induced neutrophil responses by optonisation of calcium pyrophosphate dihydrate crystals. Ann Rheum Dis 52: 599-607 (1993)]. It is supposed that shape, size and amount of the crystals play quite specific roles in PMN activation. On this subject, there are numerous studies which, while confirming the phlogogenic activity of CPPD crystals, are in poor agreement above all on the dimensions of the crystalline material able to stimulate phagocytes more intensely [Schwan et al, Schumacher H R, Fishbein P, Phelps R, Krauser R. Comparison of sodium urate and calcium pyrophosphate crystal size and other factors. Arthritis Rheum 18 (suppl): 783-793 (1995)].

At the moment, only symptomatic therapies to reduce acute pseudogout attacks are available, and they are often insufficient to have a lasting effect.

The most widely used treatment for the acute form consists of performing an arthrocentesis on the inflamed articulation, possibly associated to articular washing with physiological solution and/or local infiltration of corticosteroids [Fitzgerald R H Jr. Inrasynovial injection of steroids uses and abuses. Mayo Clin Proc 51: 655-659 (1976); Werlen D, Gabay C., Vischer T L. Corticosteroid therapy for the treatment of acute attacks of crystal-induced arthritis: an effective alternative to nonsteroidal anti-inflammatory drugs. Rev Rhum Engl Ed 63: 248-254 (1996)].

Alternatively or in association with the aforesaid therapy, non steroidal anti-inflammatory drugs and/or colchicine, although the problem of the persistence of CPPD or HAP crystals at the tissue level still remains [Abramson S B. Treatment of gout and crystal arthropathies and use and mechanisms of action of nonsteroidal anti-inflammatory drugs. Curr Opin Rheumatol 4: 295-300 (1992)].

Currently, the only prophylaxis for pseudogout attacks is the use of oral colchicine [Gonzales T, Gantes M. Prevention of acute attacks of pseudogout with oral colchicine. J Rheumatol 14: 632-633 (1987); Lange U, Schumann C, Schmidt K L. Current aspects of colchicine therapy classical indications and new therapeutic uses. Eur J Med Res 6: 150-160 (2001)]. In the case of CPPD crystals, approaches have been attempted using the enzymatic route, i.e. the enzymes that are able to degrade pyrophosphates, such as yeast phosphatase and alkaline phosphatase, although these attempts have not found a valid therapeutic application, presumably due to the difficulty of preparing adequate formulations of protein origin because of antigen problems and of the high costs of production [Xu Y, Cruz T, Cheng P T, Pritzeker K P. Effects of pyrophosphatase on dissolution of calcium pyrophosphate dihydrate crystals. J Rheumatol 18: 66-71 (1991); Shinozaki T, Xu Y, Cruz T F, Pritzeker K P. Calcium pyrophosphate dihydrate (CPPD) crystal dissolution by alkaline phosphatase: interaction of alkaline phosphatase on CPPD crystals. J Rheumatol 22: 117-123 (1995)].

Encouraging, though not definitive, results, seem to be yielded by the oral use of magnesium carbonate, with the aim of solubilizing and inhibiting the formation of CPPD crystals [Patel K J, Weidepsnul D, Palma C, Ryan L M, Walker S E. Milwaukee shoulder with massive bilateral cysts: effective therapy for hydrops of the shoulder. J Rheumatol 24: 2479-2483 (1997)].

In the literature, there are also anecdotal descriptions of the partial effectiveness of glycosaminoglycan polysulfate in the reduction of cartilage deposits of CPPD [Sarkozi A M, Nemeth-Csoka M, Bartosiewicz G. Effects of glycosaminoglycan polysulphate in the treatment of chondrocalcinosis. Clin Exp Rheumatol 6: 3-8 (1988)].

As previously mentioned, the pathogenic action of HAP crystals in the development of articular inflammatory manifestations is not quite clear, although crystalline aggregates of HAP are frequently present in articular effusions, both of inflammatory and degenerative nature, so their presence is considered an epiphenomenon. On the contrary, the action of these substances in the development of periarticular inflammatory degenerative pathologies, such as calcific periarthritis, clinically expressed in acute and/or chronic painful shoulder conditions, is well known. Currently, there are treatments aimed at the destruction and/or removal of such microcrystalline deposits such as articular washings with physiological solution and Extracorporeal Shock Wave Therapy (ESWT) [Cosentino R, De Stefano R, Selvi E, Frati E, Manca S, Frediani B, Marcolongo R. Extracorporeal Shock Wave Therapy for chronic calcific tendinitis of the shoulder: single blind study. Ann Rheum Dis 62: 248-50 (2003); Ebenbichler G R, Erdogmus C B, Resch K L, Funovics M A, Kainberger F, Barisani G, Aringer M, Nicolakis P, Wiesinger G F, Baghestanian M, Preisinger E, Fialka-Moser V. Ultrasound therapy for calcific tendinitis of the shoulder. N Engl J Med 341: 1237 (1999)].

In regard to the dissolution of HAP crystals, there are very few data in the literature, and they refer to the use of chemical substances that have no foreseeable therapeutic use [Doroshkin S V. Surface reactions of apatite dissolution. J Colloid Interface Sci 191: 489-497 (1997)].

The lack of therapeutic treatments aimed at the dissolution of the tissue deposits of CPPD and HAP, has induced the authors to research chemical principles able to dissolve the crystals present in the articular and periarticular environment.

The activity of polymetaphosphates, antagonist to the crystallization of salts based on calcium (e.g. calcium carbonate and calcium sulfate) and other metals (e.g. iron, magnesium). This class of compounds therefore finds widespread use as softeners of hard and industrial waters, detergents in textile industries and/or dispersing agents in fabric coloring operations. In cosmetics, polymetaphosphates are particularly effective in the treatment of calcareous deposits such as tartar, they are important ingredients in anti-plaque tooth pastes [Draus F. M. et al. Pyrophosphate and hexametaphosphate effects in vitro calculus formation. Archs. Oral Biol. 15: 893-896 (1970); McClanahan S. F., White D. J., Cox E. R. Dentifrice compositions containing polyphosphate and monofluorophosphate. U.S. Pat. No. 6,190,644 (2002)].

The ability of these substances to reduce aortic calcifications in rats has been demonstrated [Fleisch H, Schibler D, Maerki J, Frossard I. Inhibition of aortic calcification by means of pyrophosphate and polyphosphate. Nature 207: 1300-1301 (1965)] and skin calcification, also in rats [Schibler D, Fleisch H. Inhibition of skin calcification (calciphylaxis) by polyphosphates. Experientia 22: 367-369 (1966)] and, consequently, it is possible to consider a therapeutic use aimed at solubilizing ectopic calcifications [Irving J T, Schibler D, Fleish H. Bone formation in normal and vitamin D-treated rachitic rats during the administration of polyphosphates. Proc Soc Exp Biol Med 123: 332-335 (1966)].

The authors have already described the in vitro solubilizing ability of some polymetaphosphates on CPPD aggregates [Cini R, Chindamo D, Catenaccio M, Lorenzini S, Selvi E, Nerucci F, Picchi M P, Berti G, Marcolongo R. Dissolution of calcium pyrophosphate crystals by polyphosphates: an in vitro and ex vivo study. Ann Rheum Dis 60: 962-967 (2001)]. However, the possible limit to the clinical use of these substances derives from the fact that:

1) the same polymetaphosphates are not uniquely identified with a definite molecular weight, since their formula is (NaPO₃)ₙ, with n which may vary from 3 to over 20;
2) crystals which are partially dissolved and reduced in volume (and possibly opsonized) as a result of an increased solubility of the pyrophosphate could be phagocytosed by PMN and macrophages with increased inflammation, additional production of ROS and start of a vicious cycle that could further aggravate the pathological condition, with persistence of phlogosis [Oyanagui Y. Role of phosphate, pyrophosphate, adenine nucleotides and sulfate in activating production of the superoxide radical by macrophages, and in formation of rat paw edema. Agents Actions 7: 125:132 (1977); Swan A, Heywood B, Chapman B, Seward H, Dieppe P. Evidence for a causal relationship between the structure, size, and load of calcium pyrophosphate dihydrate crystals, and attacks of pseudogout. Ann Rheum Dis 54: 825-830 (1995); Biaglow J E, Kachur A V. The generation of hydroxyl radicals in the reaction of molecular oxygen with polyphosphate complexes of ferrous ion. Radial Res 148: 181-187 (1997)].

In the present invention, the above problems are solved thanks to the obtainment of formulations that contain polymetaphosphates with defined structure or salts thereof, which may be associated with one or more substances with anti-radical actions and/or with anti-oxidizing agents.

Therefore, the object of the invention is to provide a soluble pharmaceutical solution comprising an effective amount of at least one linear or cyclic polymetaphosphate or a soluble and pharmaceutically acceptable salt thereof, and appropriate diluents. Preferably, the salt of the polymetaphosphate is a sodic salt (NaPO₃)ₙ; more preferably, it is included in the following group: polymeric metaphosphate (SMP, formula a); tripolymetaphosphate (PSTP, formula b); cyclic trimetaphosphate (TSMP, formula c), cyclic hexametaphosphate (SEMP, formula d).

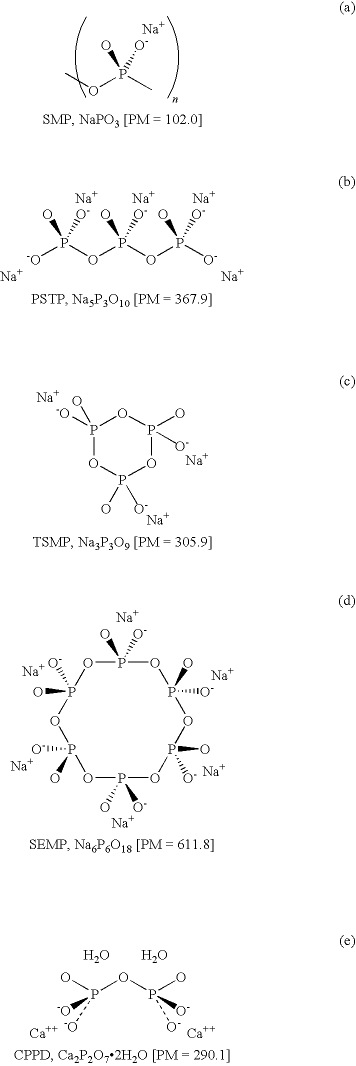

In a preferred embodiment, the composition further comprises effective quantities of anti-oxidizers and/or ROS scavengers, such as mannitol, vitamin E, vitamin C, carotenoids, tocopherol, taurine, glucosamine sulfate, glucosamine hydrochloride. To be excluded are N-acetylcysteine, glutatione. Among them, due to their effectiveness, tolerability and simplicity of preparation are to be preferred mannitol, taurine and/or glucosamine or salts thereof are to be preferred.

Mannitol is a power scavenger of oxydryl radicals [Chaturvedi V, Wong B, Newman S L. Oxidative killing of Cryptococcus neoformans by human neutrophils. Evidence that fungal mannitol protects by scavenging reactive oxygen intermediates. J Immunol 156: 3836-3840 (1996)]. Taurine is a power scavenger of the hypochlorite anion, of nitroxide radicals and of all ROS produced by PMN and/or activated macrophages [Park E, Alberti J, Quinn M R, Schuller-Levis G. Taurine chloramine inhibits the production of superoxide anion, IL-6 and IL-8 in activated human polymorphonuclear leukocytes. Adv Exp Med Biol 442: 177-182 (1998)]. Polymetaphosphate by itself is not able to solubilize the calcium-based crystals (Ca pyrophosphates, hydroxyapatite) responsible for some arthropathies, but it is an anti-oxidizing agent that acts in synergy with known anti-oxidizers, with consequent reduction of inflammatory phenomena.

In a preferred embodiment, the formulation of the invention is also associated to one or more scavenger substances.

The obtained solutions can be injected directly into the articulations, or they can be used for continuously washing said articulations, with variable concentrations both of the polymetaphosphates and of the anti-oxidizing agents, in order to favor the solubilization of the microcrystals responsible for articulation calcification, or the reduction of inflammatory “noxa”. These solutions must be isotonic, in consideration of their intra-articular use (isotony between 270 and 328 mOsmol/liter). However, it is also possible to hypothesize the use of hypo/hypertonic solutions to be used in the various therapeutic stages.

The formulation of the invention allows to inhibit the presence of ROS at the level of the articular structures produced by the phagocytosis performed by the PMN and/or macrophages at the crystalline structure level. This mechanism is responsible for oxidation stress, which is an important component of the inflammatory process, the latter being the basis for pseudogout attacks.

The formulations, in particular those containing sodium hexametaphosphate, alone or in association with anti-radicals and/or anti-oxidizers, were tested in vitro to assess the ability to solubilize synthetic CPPD crystals (both monocline and tricline). The solubilization tests on the aforesaid crystals were also conducted ex vivo on calcified meniscii removed by arthroscopic meniscectomy from patients affected by chondrocalcinosis. Moreover, cytotoxicity tests were conducted on the solutions used on cultures of human chondrocytes.

The same formulations were tested in vitro to assess their solubilizing capacity on HAP crystals as well.

Each formulation, in particular those containing also anti-radicals and anti-oxidizers, was incubated in vitro with PMN and/or macrophages to determine with the chemiluminescence method the ability to block the production of free radicals produced by appropriately stimulated PMN. Moreover, the scavenger effect on superoxide anion, the main free radical responsible for inflammatory phenomena, was evaluated as well. Another object of the invention is to provide a pharmaceutical formulation, injectable in intra-articular fashion, comprising a first container, containing the composition according to one of the claims 1 through 3 in powder form, and a second container, containing a solution of diluent in which is dissolved at least one substance with anti-radical action and/or one substance with anti-oxidizing action; the composition of the first container is dissolved before use. The volume of the formulation varies from 5 to 10 ml. The diluent solution can be used in association with polymetaphosphates or not, in order to exploit their anti-radical and anti-oxidizing action.

The formulation of the invention can also be used for the continuous washing of an articulation. In this case the volume of the formulation varies from 5 to 50 ml.

Within the scope of the invention is also a pharmaceutical containment formulation to be used after the solubilization of CPPD or HAP crystals in an articulation comprising a container containing a slightly hypotonic solution of dilutent, injectable in intra-articular fashion, in which is dissolved at least one substance with anti-radical and/or anti-oxidizing action. Containment formulations have a volume that may vary from 5 to 50 ml.

The invention shall now be described in its non limiting examples.

### EXAMPLE 1

### Preparation of Solubilizing Solutions in PBS Buffer

solutions containing polymetaphosphates, both linear and cyclic, were prepared, and pH and osmolality were measured, as shown in the following Table 1.A.

TABLE 1.A
Preparation of solubilizing solutions
with polymetaphosphates in PBS

| | Tested | | Checked |
|---|---|---|---|
| Solution | Polymetaphosphate | Preparation | parameters |
| A | Polymeric sodium | 500 mg of SMP were | pH = 6.9 |
| | metaphosphate | weighted and accurately | Isotony = 284 |
| | (SMP) | added to 100 ml of PBS | mOsm |
| | | buffer | |
| B | Linear sodium | 500 mg of PSTP were | pH = 8.7 |
| | tripolyphosphate | weighted and accurately | Isotony = 300 |
| | (PSTP) | added to 100 ml of PBS | mOsm |
| | | buffer | |
| C | Cyclic sodium | 500 mg of TSMP were | pH = 7.3 |
| | trimetaphosphate | weighted and accurately | Isotony = 314 |
| | (TSMP) | added to 100 ml of PBS | mOsm |
| | | buffer | |
| D | Cyclic sodium | 500 mg of SEMP were | pH = 7.0 |
| | hexametaphosphate | weighted and accurately | Isotony = 285 |
| | (SEMP) | added to 100 ml of PBS | mOsm |
| | | buffer | |

### EXAMPLE 2

### Measurement of Solubilizing Activity on CPPD Crystals

### Description of the Solubilization Procedure and Method of Analysis

5 mg of synthetic CPPD crystals, both tricline and monocline (with average size 1-30 μm) were added to 5 ml of phosphate buffer without Ca²⁺ and Mg²⁺ (PBS) containing w different types of polymetaphosphate at the concentration of 5 mg/ml (the four solutions mentioned in Table 1.A).

The suspension was maintained at 37° C. for 1 hour under continuous agitation and subsequently filtered through 0.22 μm filters. The filtrates were subjected to analysis with spectrophotometry in atomic absorption for measurements of the final calcium concentration and the percentage of dissolution of CPPD crystals was calculated based on this data.

### Solubilization Results and Conclusions

The results obtained can be summarized in the following Table 2.A.

TABLE 2.A
Solubilizing effect on CPPD crystals after
1 hour of incubation at 37° C. in PBS

| | Polymetaphosphate | Dissolution | % of |
|---|---|---|---|
| Solution | (5 mg/ml) | (mg of CPPD/ml) | dissolution |
| a | Polymeric sodium | 0.344 (12) | 27.5 |
| | metaphosphate (SMP) | | |
| b | Linear sodium | 0.310 (11) | 24.7 |
| | tripolyphosphate (PSTP) | | |
| c | Cyclic sodium | 0.023 (5) | 1.9 |
| | trimetaphosphate (TSMP) | | |
| d | Cyclic sodium | 0.461 (12) | 55.4 |
| | hexametaphosphate (SEMP) | | |

The results show that the solubilizing power of the examined polymetaphosphates on CPPD microcrystals can be expressed in the following order: SEMP>SMP>PSTP>TSMP.

Sodium hexametaphosphate has the greatest solubilizing activity on calcium pyrophosphate, whereas cyclic sodium trimetaphosphate has practically no solubilizing capacity.

The solubilizing capacity of sodium hexametaphosphate (SEMP) was then measured also as a function of time, observing the percentage of dissolution of CPPD at 15, 30 and 60 minutes at 37° C. The results are shown in table 2.B.

TABLE 2.B
Profile of the dissolving capacity of SEMP (5 mg/ml) on
CPPD crystals after progressively greater time intervals.

| Time | Dissolution | |
|---|---|---|
| Minutes | (expressed in | % of |
| (37° C.) | mg of CPPD/ml) | dissolution |
| 15 | 0.423 | 50.8 |
| 30 | 0.451 | 54.0 |
| 60 | 0.461 | 55.4 |

The effect of sodium hexametaphosphate therefore appears to be rapid, with relevant dissolution already at 15 minutes. This results indicate a possible intra-articular use of this solution for CPPD solubilization (point number 4 of the achieved results).

### EXAMPLE 3

### Solubilizing Effect on HAP Crystals

### Description of the Solubilization Procedure and Analysis Method

With a method similar to the preceding example (using 8 mg of HAP crystals), the dissolving capacities of the formulations described in Table 1.A were also studied on synthetic microcrystals of HAP (10-20 μm).

### Solubilization Results and Conclusions

The results obtained can be summarized in the following Table 3.A

TABLE 3.A
Solubilizing effect on HAP crystals after
1 hour of incubation at 37° C. in PBS

| | | Dissolution | |
|---|---|---|---|
| | Polymetaphosphate | (expressed in | % of |
| Solution | (5 mg/ml) | mg of HAP/ml) | dissolution |
| a | Polymeric sodium | 0.288 (11) | 18.0 |
| | metaphosphate (SMP) | | |
| d | Cyclic sodium | 0.150 (9) | 10.0 |
| | hexametaphosphate (SEMP) | | |

The results snow that capacity on HAP crystals is greater for SMP than for SEMP. In this case, as well, the values are relatively high and such as to program continuous washing procedures on articulations containing HAP calcifications.

The solubilizing capacity of polymeric sodium metaphosphate (SMP) was then measured as a function of time (as in the preceding example) and the results are summarized in Table 3.B.

TABLE 3.B
Profile of the dissolving capacity of SMP (5 mg/ml) on HAP
crystals after progressively greater time intervals.

| Time | Dissolution | |
|---|---|---|
| Minutes | (expressed in | % of |
| (37° C.) | mg of HAP/ml) | dissolution |
| 15 | 0.273 (11) | 17.0 |
| 30 | 0.296 (12) | 18.5 |
| 60 | 0.288 (11) | 18.0 |

This result shows that a relevant dissolution is also reached after a short time (15 minutes) if compared to the maximum dissolution achieved after longer times.

### EXAMPLE 4

### Check of Cytotoxic Effect on Chondrocytes

### Description of the Cytotoxicity Test

Samples of articular cartilage were obtained from the femoral heads of osteoarthritis patients subjected to hip prosthetization. Immediately after removal, portions of healthy cartilage were removed aseptically and 2 mm² fragments were washed in physiological solution with antibiotics, then digested with 1 mg/ml of clostridial collagenase in PBS with antibiotics for 14-18 hours at 37° C. with moderate agitation. The solution was then filtered, washed in physiological solution and centrifuged. About 90-95% of the chondrocytes were found to be vital with the method of the Trypan blue vital dye, then pre-washed and left in plates with suitable culture medium at 37° C. and 5% of CO₂.

The cells thus obtained were incubated with progressively greater concentrations of polymetaphosphates in PBS (pH 7.4) for 24 hours (6 wells for each tested concentration). The control culture was obtained incubating cells with PBS alone for 24 hours.

Cytotoxicity was determined after 1 day of exposure both with polymeric sodium metaphosphate (SMP) and with cyclic sodium hexametaphosphate (SEMP) with the tetrazole salt (MTT) method. In parallel, human chondrocytes incubated for 24 hours both with SMP and with SEMP were removed from the wells, washed in PBS; centrifuged and then fixed for 2 hours at 4° C. with Kamovsky's fixative, washed in cacodilate buffer and post-fixed for one hour at 4° C. with 1% of buffered osmium oxide, dehydrated and then included in resin to be subjected to sectioning with ultramicrotome. About 30 chondrocytes for each patient were examined with an electronic microscope.

### Results of the Cytotoxic Effect and Conclusions

The results are summarized in the following Table 4.A.

TABLE 4.A
Cytotoxic effect of growing concentrations of polymetaphosphates
(SMP or SEMP) on human chondrocytes with the MTT method

| | 0 | 1 | 2 | 5 | 15 |
|---|---|---|---|---|---|
| | SMP Solutions (mg/ml) | | | | |
| % of metabol- | 100 | 95.0 ± 3.2 | 92.8 ± 4.0 | 63.2 ± 5.1 | 50.0 ± 7.6 |
| ically active | | | | | |
| cells (mean ± SD) | | | | | |
| | SEMP Solution (mg/ml) | | | | |
| % of metabol- | 100 | 86.7 ± 4.6 | 85.2 ± 6.8 | 68.0 ± 5.2 | 48.3 ± 8.4 |
| ically active | | | | | |
| cells (mean ± SD) | | | | | |

Values are expressed as the mean ± SD in 4 separate experiments.

The results show that the 50% inhibitory dose was reached at the highest tested concentration (15 mg/ml). In no case did morphological evaluation with the electronic microscope show cell structure alteration.

### EXAMPLE 5

### SEM and SEMP Based Formulations, Associated to Components with Anti-Radical and/or Anti-Oxidizing Activity

Pharmaceutical Formulations of SEMP with Anti-ROS

Several pharmaceutical formulations were prepared, composed by cyclic sodium hexametaphosphate with different compounds that have ROS and hypochlorite anion scavenging capacity.

The CPPD crystal solubilizing capacity of each selected formulation was checked, to verify whether the presence of anti-oxidizing and/or anti-radical substances could inhibit the solubilization of pyrophosphate salts.

The pharmaceutical formulations are set out below:

| Components | Concentration % (w/v) |
|---|---|
| Formulation A | |
| Cyclic sodium hexametaphosphate | 1.5 |
| Monobasic potassium phosphate | 0.04 |
| Potassium chloride | 0.04 |
| Dibasic sodium phosphate | 0.23 |
| Sodium chloride | 0.65 |
| Isotony mOsm | 297 |
| pH | 7.5 |
| Appearance | clear |
| Formulation B | |
| Cyclic sodium hexametaphosphate | 0.75 |
| Monobasic potassium phosphate | 0.06 |
| Potassium chloride | 0.06 |
| Dibasic sodium phosphate | 0.345 |
| Mannitol | 3.17 |
| Taurine | 0.3 |
| Isotony mOsm | 292 |
| pH | 7.5 |
| Appearance | clear |
| Formulation D | |
| Cyclic sodium hexametaphosphate | 0.75 |
| Monobasic potassium phosphate | 0.06 |
| Potassium chloride | 0.06 |
| Dibasic sodium phosphate | 0.345 |
| Glucosamine sulfate | 2.20 |
| Isotony mOsm | 310 |
| pH | 6.7 |
| Appearance | clear |
| Formulation O | |
| Cyclic sodium hexametaphosphate | 0.5 |
| Monobasic potassium phosphate | 0.12 |
| Potassium chloride | 0.12 |
| Dibasic sodium phosphate | 0.69 |
| Mannitol | 1.55 |
| Taurine | 0.3 |
| Isotony mOsm | 290 |
| pH | 7.3 |
| Appearance | clear |
| Formulation F | |
| Cyclic sodium hexametaphosphate | 0.5 |
| Monobasic potassium phosphate | 0.06 |
| Potassium chloride | 0.06 |
| Dibasic sodium phosphate | 0.345 |
| Mannitol | 3.17 |
| Glucosamine sulfate | 0.4 |
| Isotony mOsm | 304 |
| pH | 7.0 |
| Appearance | clear |
| Formulation L | |
| Cyclic sodium hexametaphosphate | 0.5 |
| Monobasic potassium phosphate | 0.1 |
| Potassium chloride | 0.1 |
| Dibasic sodium phosphate | 0.575 |
| Mannitol | 2.64 |
| N-acetylcysteine | 0.32 |
| Isotony mOsm | 302 |
| pH | 6.7 |
| Appearance | clear |
| Formulation N | |
| Cyclic sodium hexametaphosphate | 0.5 |
| Monobasic potassium phosphate | 0.12 |
| Potassium chloride | 0.12 |
| Dibasic sodium phosphate | 0.69 |
| Mannitol | 1.55 |
| Taurine | 0.3 |
| N-acetylcysteine | 0.32 |
| Isotony mOsm | 297 |
| pH | 6.6 |
| Appearance | Clear |

Pharmaceutical Formulations of SMP with Anti-ROS

| Components | Concentration % (w/v) |
|---|---|
| Formulation A1 | |
| Polymeric sodium metaphosphate | 1.5 |
| Monobasic potassium phosphate | 0.04 |
| Potassium chloride | 0.04 |
| Dibasic sodium phosphate | 0.23 |
| Sodium chloride | 0.65 |
| Isotony mOsm | 295 |
| pH | 7.4 |
| Appearance | clear |
| Formulation B1 | |
| Polymeric sodium metaphosphate | 0.75 |
| Monobasic potassium phosphate | 0.06 |
| Potassium chloride | 0.06 |
| Dibasic sodium phosphate | 0.345 |
| Mannitol | 3.17 |
| Taurine | 0.3 |
| Isotony mOsm | 290 |
| pH | 7.4 |
| Appearance | clear |
| Formulation D1 | |
| Polymeric sodium metaphosphate | 0.75 |
| Monobasic potassium phosphate | 0.06 |
| Potassium chloride | 0.06 |
| Dibasic sodium phosphate | 0.345 |
| Glucosamine sulfate | 2.20 |
| Isotony mOsm | 308 |
| pH | 6.6 |
| Appearance | clear |
| Formulation O1 | |
| Polymeric sodium metaphosphate | 0.5 |
| Monobasic potassium phosphate | 0.12 |
| Potassium chloride | 0.12 |
| Dibasic sodium phosphate | 0.69 |
| Mannitol | 1.55 |
| Taurine | 0.3 |
| Isotony mOsm | 287 |
| pH | 7.2 |
| Appearance | clear |
| Formulation F1 | |
| Polymeric sodium metaphosphate | 0.5 |
| Monobasic potassium phosphate | 0.06 |
| Potassium chloride | 0.06 |
| Dibasic sodium phosphate | 0.345 |
| Mannitol | 3.17 |
| Glucosamine sulfate | 0.4 |
| Isotony mOsm | 300 |
| pH | 6.9 |
| Appearance | clear |
| Formulation L1 | |
| Polymeric sodium metaphosphate | 0.5 |
| Monobasic potassium phosphate | 0.1 |
| Potassium chloride | 0.1 |
| Dibasic sodium phosphate | 0.575 |
| Mannitol | 2.64 |
| N-acetylcysteine | 0.32 |
| Isotony mOsm | 299 |
| pH | 6.5 |
| Appearance | clear |
| Formulation N1 | |
| Polymeric sodium metaphosphate | 0.5 |
| Monobasic potassium phosphate | 0.12 |
| Potassium chloride | 0.12 |
| Dibasic sodium phosphate | 0.69 |
| Mannitol | 1.55 |
| Taurine | 0.3 |
| N-acetylcysteine | 0.32 |
| Isotony mOsm | 295 |
| pH | 6.5 |
| Appearance | clear |

### Check of Solubilizing Capacity on CPPD Crystals

The aforesaid formulations O, F, L, N containing SEMP with different compounds having anti-radical and anti-oxidizing activity were evaluated for their solubilizing capacity on CPPD crystals.

The pharmaceutical formulations O and F, containing SEMP respectively with mannitol+taurine and with mannitol+glucosamine sulfate, were found to be active in the solubilization of CPPD crystals, as shown by the results set out in the following Table 5.A.

TABLE 5.A
Solubilizing effect on CPPD crystals (Formulations O and F)

| Incubation time | Dissolution (expressed | |
|---|---|---|
| (in minutes at 37° C.) | in mg of CPPD/ml) | % of dissolution |
| 15 | 0.527 | 53.1 |
| 30 | 0.552 | 57.2 |
| 60 | 0.577 | 62.4 |

The pharmaceutical formulations L and N, containing SEMP respectively with mannitol+taurine+N-acetylcysteine and with mannitol+N-acetylcysteine, were found to be inactive in the solubilization of CPPD crystals, as the dissolving medium almost completely loses its potential with respect to CPPD crystals and the concentration of calcium in the filtrate is below the limit of receivability of the technique employed.

The aforesaid formulations O1, F1, L1, N1, containing SMP with different compounds having anti-radical and/or anti-oxidizing activity were evaluating for their solubilizing capacity on CPPD crystals.

The pharmaceutical formulations O1 and F1, containing SMP respectively with mannitol+taurine and with mannitol+glucosamine sulfate, were found to be active in the solubilization of CPPD crystals, as shown by the results set out in the following Table 5.B.

TABLE 5.B
Solubilizing effect on CPPD crystals (Formulations O1 and F1)

| Incubation time | Dissolution (expressed | |
|---|---|---|
| (in minutes at 37° C.) | in mg of CPPD/ml) | % of dissolution |
| 15 | 0.189 | 20.5 |
| 30 | 0.214 | 23.2 |
| 60 | 0.254 | 27.5 |

The above results are surprising because they show that the selection of anti-oxidizing and anti-radical agents must be careful. For example, the presence of a power anti-oxidizer, such as N-acetylcysteine, can drastically reduce the solubilizing effect of polyphosphates.

### Check of Solubilizing Capacity on HAP Crystals

The aforementioned formulations O, F, L, N containing SEMP with different compounds having anti-radical and anti-oxidizing activity were evaluated for their solubilizing capacity on HA crystals.

The pharmaceutical formulations O and F, containing SEMP respectively with mannitol+taurine and with mannitol+glucosamine sulfate, were found to be active in the solubilization of HA crystals, as shown by the results set out in the following Table 5.C.

TABLE 5.C
Solubilizing effect on HAP crystals (Formulations O and F)

| Incubation time | Dissolution (expressed | |
|---|---|---|
| (in minutes at 37° C.) | in mg of CPPD/ml) | % of dissolution |
| 15 | 0.128 | 8.4 |
| 30 | 0.134 | 8.9 |
| 60 | 0.150 | 10.0 |

The pharmaceutical formulations L and N, containing SEMP respectively with mannitol+taurine+N-acetylcysteine and with mannitol+N-acetylcysteine, were found to be inactive in the solubilization of HAP crystals, as the dissolving medium almost completely loses its potential with respect to HAP crystals and the concentration of calcium in the filtrate is below the limit of receivability of the technique employed.

The aforesaid formulations O1, F1, L1, N1, containing SMP with different compounds having anti-radical and/or anti-oxidizing activity were evaluating for their solubilizing capacity on HA crystals.

The pharmaceutical formulations O1 and F1, containing SMP respectively with mannitol+taurine and with mannitol+glucosamine sulfate, were found to be active in the solubilization of HA crystals, as shown by the results set out in the following Table 5.D.

TABLE 5.D
Solubilizing effect on HAP crystals (Formulations O1 and F1)

| Incubation time | Dissolution (expressed | |
|---|---|---|
| (in minutes at 37° C.) | in mg of HAP/ml) | % of dissolution |
| 15 | 0.121 | 8.1 |
| 30 | 0.127 | 8.5 |
| 60 | 0.136 | 9.1 |

In the case of the solubilization of HA crystals, too, the selection of anti-oxidizing and anti-radical agents must be careful. For example, the presence of a power anti-oxidizer, such as N-acetylcysteine, practically eliminates the solubilizing effect of polyphosphates.

### EXAMPLE 6

### Measurement of Anti-Radical and/or Anti-Oxidizing

Tested Pharmaceutical Formulations of SEMP with Anti-ROS

| | Concentration % |
|---|---|
| | (w/v) |
| Formulation A | |
| Components | |
| Cyclic sodium | 1.5 |
| hexametaphosphate | |
| Monobasic potassium phosphate | 0.04 |
| Potassium chloride | 0.04 |
| Dibasic sodium phosphate | 0.23 |
| Sodium chloride | 0.65 |
| Isotony mOsm | 297 |
| pH | 7.5 |
| Appearance | clear |
| Formulation B | |
| Components | |
| Cyclic sodium | 0.75 |
| hexametaphosphate | |
| Monobasic potassium phosphate | 0.06 |
| Potassium chloride | 0.06 |
| Dibasic sodium phosphate | 0.345 |
| Mannitol | 3.17 |
| Taurine | 0.3 |
| Isotony mOsm | 292 |
| pH | 7.5 |
| Appearance | clear |
| Formulation D | |
| Components | |
| Cyclic sodium | 0.75 |
| hexametaphosphate | |
| Monobasic potassium phosphate | 0.06 |
| Potassium chloride | 0.06 |
| Dibasic sodium phosphate | 0.345 |
| Glucosamine sulfate | 2.20 |
| Isotony mOsm | 310 |
| pH | 6.7 |
| Appearance | clear |
| Formulation E | |
| Components | |
| Monobasic potassium phosphate | 0.08 |
| Potassium chloride | 0.08 |
| Dibasic sodium phosphate | 0.46 |
| Glucosamine sulfate | 2.20 |
| Isotony mOsm | 312 |
| pH | 6.9 |
| Appearance | clear |
| Formulation F | |
| Components | |
| Cyclic sodium | 0.5 |
| hexametaphosphate | |
| Monobasic potassium phosphate | 0.06 |
| Potassium chloride | 0.06 |
| Dibasic sodium phosphate | 0.345 |
| Mannitol | 3.17 |
| Glucosamine sulfate | 0.4 |
| Isotony mOsm | 304 |
| pH | 7.0 |
| Appearance | clear |
| Formulation G | |
| Components | |
| Monobasic potassium phosphate | 0.08 |
| Potassium chloride | 0.08 |
| Dibasic sodium phosphate | 0.46 |
| Mannitol | 3.17 |
| Glucosamine sulfate | 0.4 |
| Isotony mOsm | 302 |
| pH | 7.2 |
| Appearance | clear |
| Formulation O | |
| Components | |
| Cyclic sodium | 0.5 |
| hexametaphosphate | |
| Monobasic potassium phosphate | 0.12 |
| Potassium chloride | 0.12 |
| Dibasic sodium phosphate | 0.69 |
| Mannitol | 1.55 |
| Taurine | 0.3 |
| Isotony mOsm | 290 |
| pH | 7.3 |
| Appearance | clear |

Tested Pharmaceutical Formulations of SMP with Anti-ROS

| | Concentration % |
|---|---|
| | (w/v) |
| Formulation A1 | |
| Components | |
| Sodium metaphosphate | 1.5 |
| Monobasic potassium phosphate | 0.04 |
| Potassium chloride | 0.04 |
| Dibasic sodium phosphate | 0.23 |
| Sodium chloride | 0.65 |
| Isotony mOsm | 295 |
| pH | 7.4 |
| Appearance | clear |
| Formulation B1 | |
| Components | |
| Sodium metaphosphate | 0.75 |
| Monobasic potassium phosphate | 0.06 |
| Potassium chloride | 0.06 |
| Dibasic sodium phosphate | 0.345 |
| Mannitol | 3.17 |
| Taurine | 0.3 |
| Isotony mOsm | 290 |
| pH | 7.4 |
| Appearance | clear |
| Formulation C1 | |
| Components | |
| Monobasic potassium phosphate | 0.02 |
| Potassium chloride | 0.02 |
| Dibasic sodium phosphate | 0.115 |
| Mannitol | 5.17 |
| Taurine | 0.3 |
| Isotony mOsm | 304 |
| pH | 7.4 |
| Appearance | clear |
| Formulation D1 | |
| Components | |
| Sodium metaphosphate | 0.75 |
| Monobasic potassium phosphate | 0.06 |
| Potassium chloride | 0.06 |
| Dibasic sodium phosphate | 0.345 |
| Glucosamine sulfate | 2.20 |
| Isotony mOsm | 308 |
| pH | 6.6 |
| Appearance | clear |
| Formulation E1 | |
| Components | |
| Monobasic potassium phosphate | 0.08 |
| Potassium chloride | 0.08 |
| Dibasic sodium phosphate | 0.46 |
| Glucosamine sulfate | 2.20 |
| Isotony mOsm | 310 |
| pH | 6.8 |
| Appearance | clear |
| Formulation F1 | |
| Components | |
| Sodium metaphosphate | 0.5 |
| Monobasic potassium phosphate | 0.06 |
| Potassium chloride | 0.06 |
| Dibasic sodium phosphate | 0.345 |
| Mannitol | 3.17 |
| Glucosamine sulfate | 0.4 |
| Isotony mOsm | 302 |
| pH | 6.9 |
| Appearance | clear |
| Formulation G1 | |
| Components | |
| Monobasic potassium phosphate | 0.08 |
| Potassium chloride | 0.08 |
| Dibasic sodium phosphate | 0.46 |
| Mannitol | 3.17 |
| Glucosamine sulfate | 0.4 |
| Isotony mOsm | 300 |
| pH | 7.1 |
| Appearance | clear |
| Formulation O1 | |
| Components | |
| Polymeric sodium | 0.5 |
| metaphosphate | |
| Monobasic potassium phosphate | 0.12 |
| Potassium chloride | 0.12 |
| Dibasic sodium phosphate | 0.69 |
| Mannitol | 1.55 |
| Taurine | 0.3 |
| Isotony mOsm | 287 |
| pH | 7.2 |
| Appearance | clear |

### Procedure for Chemiluminescence Produced by Human PMNs

Chemiluminescence [De Luca M A, McElroy W D. Bioluminescence and chemiluminescence. Methods in Enzymol 133: 449-493 (1986)] is a method to evaluate the scavenger action on the pool of the ROS produced by polymorphonucleates (PMN) stimulated with zymosan [10 mg/ml of phosphate buffer without Ca²⁺ and Mg²⁺ (PBS); Sigma] opsonized according to the English method [English D, Roloff J S, Lukens J N. Regulation of human polymorphonuclear leucocyte superoxide release by cellular response to chemotactic peptides. J Immun 126: 165-171 (1981)]. The PMNs were obtained from samples of peripheral venous blood of healthy subjects by centrifuging in density gradient: polymorphoprep (Nycomed), which, once centrifuged, forms a density gradient whereon the blood cells are separated.

The purity (>90%) and the vitality (>95%) of the cell population were tested by examining a strip and conducting the trypan blue exclusion test. Thereafter, to a portion (100 μl) of a suspension containing 10⁶ PMN ml⁻¹ of PBS, were added 100 μl of luminol (2 mg in 10 ml of NaOH 0.01M subsequently diluted 1:10 with PBS) and 10 μl of stimulator. The preparation was introduced in the chemiluminometer (Berthold Multi-biolumat LB 9505C) at 37° C.; the reaction kinetics were read for 40 minutes. All cpm values shown in the tables are extrapolated from an average of 2 values (double analysis).

For each experiment, three distinct trials were conducted.

### Inhibition Test of the Chemiluminescence Produced by Human PMNs Relating to Solutions Containing SEMP in the Presence or Absence of Other Anti-Oxidizing Substances

The results were collected in the following Table 6.A

TABLE 6.A
Effect on chemiluminescence of formulations containing
SEMP and anti-oxidants

| Formulation | Test 1 | Test 2 | Test 3 |
|---|---|---|---|
| Basal | % inhibition | % inhibition | % inhibition |
| A | 79.4 | 77.3 | 80.2 |
| B | 77.9 | 75.5 | 77.1 |
| C | 7.0 | 7.7 | 8.3 |
| D | 94.5 | 92.9 | 94.4 |
| E | 86.9 | 82.1 | 86.9 |
| F | 96.9 | 87.7 | 91.2 |
| G | 66.3 | 64.5 | 74.1 |
| O | 56.5 | 65.7 | 66.6 |

NOTE:
the formulations C (taurine and mannitol), G (glucosamine and mannitol) and E (glucosamine) do not contain SEMP.

The results of the inhibition of chemiluminescence due to scalar quantities of sodium hexametaphosphate, without anti-oxidants, are instead shown in the following Table 6.B.

TABLE 6.B
Effect of scalar quantities of SEMP sodium (alone) on
chemiluminescence

| Concentration of | | | |
|---|---|---|---|
| SEMP in PBS | Test 1 | Test 2 | Test 3 |
| (mg/ml) | % | % | % |
| Basal | inhibition | inhibition | inhibition |
| 0.5 | 32.4 | 17.5 | 33.4 |
| 1 | 64.8 | 50.0 | 66.9 |
| 2 | 74.6 | 72.5 | 70.0 |
| 4 | 81.0 | 80.0 | 74.3 |
| 7.5 | 97.8 | 84.0 | 76.9 |

All tested formulations have shown a powerful inhibitory effect on the chemiluminescence produced by human PMNs with the procedure described above. The most amazing and unexpected was that simple solutions of sodium hexametaphosphate in PBS have shown a powerful inhibiting effect on chemiluminescence. The addition of known anti-oxidants and/or anti-radical agents allowed to maintain the inhibitory effect on chemiluminescence.

Moreover, the formulations C, E, G which do not contain SEMP must be considered the formulations for containment or rather for washing the articulation after intervening with the solutions containing sodium hexametaphosphate. These solutions must be considered as an instrument for treating chondrocalcinosis and hence for the prophylaxis of pseudogout episodes.

### Test of Inhibition of the Chemiluminescence Produced by Human PMNs Relating to Solutions Containing SMP in the Presence or Absence of Other Anti-Oxidizing Substances

TABLE 6.C
Effect on chemiluminescence of formulations containing
SMP and anti-oxidants

| Formulation | Test 1 | Test 2 | Test 3 |
|---|---|---|---|
| Basal | % inhibition | % inhibition | % inhibition |
| A1 | 75.9 | 72.5 | 75.0 |
| B1 | 92.5 | 90 | 91.5 |
| D1 | 84.9 | 80.1 | 83.9 |
| F1 | 54.3 | 62.5 | 72.5 |
| O1 | 77.4 | 75.0 | 78.5 |

The results of the inhibition of chemiluminescence due to scalar quantities of polymeric sodium hexametaphosphate, without anti-oxidants, are instead shown in the following Table 6.D.

TABLE 6.D
Effect of scalar quantities of SMP sodium (alone)
on chemiluminescence

| Concentration of | | | |
|---|---|---|---|
| SEMP in PBS | Test 1 | Test 2 | Test 3 |
| (mg/ml) | % | % | % |
| Basal | inhibition | inhibition | inhibition |
| 0.5 | 42.5 | 52.8 | 34 |
| 1 | 69.1 | 70 | 70 |
| 2 | 77.6 | 70 | 73.6 |
| 4 | 79.8 | 76 | 79.2 |
| 7.5 | 82 | 75 | 81.5 |

Formulations containing SMP have also shown a powerful inhibitory effect on the chemiluminescence produced by human PMNs with the procedure described above, with results which may be superposed with those already observed with hexametaphosphate.

### EXAMPLE 7

### Effect on the Vitality of Human Polymorphonucleates (PMN)

### Method for Determining PMN Vitality

The solutions were prepared solubilizing the sodium hexametaphosphate in PBS and adding PMNs (1×10⁵/ml), obtained from venous blood of healthy volunteers. Incubation was performed at 37° C. for 5 minutes. Subsequently, Trypan was added and the cells were observed with the microscope, calculating the number of vital cells.

Tests with SEMP

The vitality of the PMNs in contact with solutions containing scalar quantities of sodium hexametaphosphate was tested, in the presence or absence of the same anti-oxidants and/or anti-radical agents for chemiluminescence inhibition tests. For each concentration, pH and osmolality were measured as well (the pH of all solutions was brought back to 7.5). The results are shown in Table 7.A.

TABLE 7.A

| Concentration of | | Osmolality | % |
|---|---|---|---|
| SEMP in PBS (mg/ml) | pH | (mOsm) | Vitality PMN |
| 0.5 | 7.5 | 273 | 100 |
| 1 | 7.5 | 274 | 97 |
| 2 | 7.5 | 274 | 96 |
| 4 | 7.5 | 280 | 92 |
| 7.5 | 7.5 | 294 | 80 |
| 15.0 | 7.5 | 322 | 75 |

None of the tested concentrations caused a marked reduction in PMN vitality, except for the maximum tested concentration (15 mg/ml).

The experiment was repeated using formulations containing hexametaphosphate and various anti-oxidants (see Example 6), without harmful effects on PMN survival. The results are shown in Table 7.B.

TABLE 7.B

| | | Osmolality | % |
|---|---|---|---|
| Formulation | pH | (mOsm) | Vitality PMN |
| A | 7.5 | 297 | 98 |
| B | 7.5 | 292 | 99 |
| C | 7.5 | 306 | 98 |
| D | 6.7 | 310 | 98 |
| E | 6.9 | 312 | 97 |
| F | 7.0 | 304 | 93 |
| G | 7.2 | 302 | 98 |
| L | 6.6 | 302 | 91 |
| N | 6.6 | 297 | 97 |
| O | 7.3 | 290 | 97 |

Tests with SMP

The vitality of the PMNs in contact with solutions containing scalar quantities of sodium metaphosphate was tested, in the presence or absence of the same anti-oxidants and/or anti-radical agents for chemiluminescence inhibition tests. For each concentration, pH and osmolality were measured as well (the pH of all solutions was brought back to 7.5). The results are shown in Table 7.C.

TABLE 7.C

| Concentration of | | Osmolality | % |
|---|---|---|---|
| SEMP in PBS (mg/ml) | pH | (mOsm) | Vitality PMN |
| 0.5 | 7.5 | 268 | 99 |
| 1 | 7.5 | 269 | 97 |
| 2 | 7.5 | 271 | 98 |
| 4 | 7.5 | 282 | 93 |
| 7.5 | 7.5 | 292 | 84 |
| 15.0 | 7.5 | 320 | 74 |

None of the tested concentrations caused a marked reduction in PMN vitality, except for the maximum tested concentration (15 mg/ml).

The experiment was repeated using formulations containing metaphosphate and anti-oxidants (see Example 6), without harmful effects on PMN survival. The results are shown in Table 7.B.

TABLE 7.B

| | | Osmolality | % |
|---|---|---|---|
| Formulation | pH | (mOsm) | Vitality PMN |
| A1 | 7.4 | 295 | 96 |
| B1 | 7.4 | 290 | 97 |
| C1 | 7.4 | 304 | 99 |
| D1 | 6.6 | 308 | 95 |
| E1 | 6.8 | 310 | 98 |
| F1 | 6.9 | 302 | 90 |
| G1 | 7.1 | 300 | 98 |
| L1 | 6.5 | 299 | 88 |
| N1 | 6.5 | 295 | 96 |
| O1 | 7.2 | 287 | 94 |

### EXAMPLE 8

### Measurement of Superoxide Anion Inhibition

### Method for Determining Superoxide Anion

The production of O₂ by stimulated PMNs [in this case, stimulation was conducted with Phorbol 12-myristate 13-acetate (PMA)], was evaluated through the reduction of the cytochrome-C, as described in English's method [English D, Roloff J S, Lukens J N. Regulation of human polymorphonuclear leucocyte superoxide release by cellular response to chemotattic peptides. J Immun 126: 165-171 (1981)]. For this purpose, to a portion of 750 μl of PBS were added, in this order: 100 μl of cytochrome-C (30 mg/ml), 100 μl of stimulator and 100 μl of cellular suspension. The preparation was incubated for 25 minutes at 37° C.; subsequently, 50 μl of superoxide dismutase (SOD) 1 mg/ml, 75000 units (Sigma) to stop the reaction, lastly centrifuging for 10 minutes at 4° C. and a spectrophotometric reading (Beckman DU6) of the surnatant at 550 and 468 nm. The “white” was prepared introducing the SOD in a sample before all other reactants. The PMNs were prepared as described previously, the stimulator (PMA) was prepared as described in English's method. The results are expressed in nMoles/10⁶ PMNs.

It is interesting to note that the scavenger effect on superoxide anion is directly proportional to the concentration of only hexametaphosphate in PBS and it is readily apparent at the concentration of 5 mg/ml. The addition of anti-oxidants like mannitol and taurine (Formulation O with 0.5 mg/ml SEMP) considerably modified the anti-oxidizing activity of hexametaphosphate, alone at equal concentration.

Tests with SEMP

The results are summarized in Table 8.A

TABLE 8.A

| Table 8.A | | |
|---|---|---|
| Formulations | Test 1 | Test 2 |
| Basal | % inhibition | % inhibition |
| PBS + SEMP 0.5 mg/ml | 12.5 | 14.0 |
| PBS + SEMP 1 mg/ml | 30.8 | 38.8 |
| PBS + SEMP 2 mg/ml | 43.7 | 47.4 |
| PBS + SEMP 5 mg/ml | 53.1 | 56.2 |
| Formulation O | 78.6 | 74.7 |
| Formulation E | 75 | 70 |
| Formulation G | 69.7 | 79.7 |

Unexpectedly, hexametaphosphate showed an inhibitory power on the production of superoxide anion, in direct proportion to its concentration. The presence of other anti-oxidizing or anti-radical substances enhances said inhibiting effect.

The experiment of the superoxide anion show, more than was already demonstrated by the chemiluminescence experiment, the extreme importance from the therapeutic viewpoint and the high degree of innovation from the patent viewpoint, of the association of polymetaphosphates with anti-oxidizing and/or anti-radical substances.

Moreover, the formulations C, E and G can also be considered the formulations for the containment or rather the washing of the articulation after intervening with solutions containing sodium hexametaphosphate. It can be considered as a point reached for containment solutions.

1. Use of a linear or cyclic polymetaphosphate or a soluble salt thereof for the preparation of an intra-articular injectable medicament for the treatment of articular pathologies.

2. Use according to claim 1 wherein the soluble salt is the sodic salt.

3. Use according to claim 1 wherein the polymetaphosphate is included in the following group: polymeric metaphosphate (SMP); tripolymetaphosphate (PSTP); cyclic trimetaphosphate (TSMP), cyclic hexametaphosphate (SEMP).

4. Use according to claim 1 wherein the medicament further comprises effective amounts of anti-oxidants and/or anti-radicals of oxygen and hypochlorite anion.

5. Use according to claim 4 wherein the anti-oxidants are included in the following group: mannitol, vitamin E, vitamin C, carotenoids, tocopherol, taurine, glucosamine sulfate, glucosamine hydrochloride.

6. Use according to any of the previous claims, wherein the medicament further comprises at least one scavenger substance with anti-radical activity.

7. Use according to claim 1 wherein the articular pathology is characterized by calcium pyrophosphate dehydrate (CPPD) and/or hydroxyapatite HAP intra-articular deposits.

8. Use according to claim 1 wherein the medicament has an antioxydant activity.

9. A soluble pharmaceutical composition comprising pharmaceutically effective amounts of cyclic sodium hexametaphosphate or polymeric sodium metaphosphate, mannitol and taurine.

10. Composition according to claim 9 in which the amount of cyclic sodium hexametaphosphate or polymeric sodium metaphosphate is at least 0.5% (w/v).

11. Composition according to claim 9 in which the amount of mannitol is 1.55% (w/v).

12. Composition according to claim 9 in which the amount of taurine is 0.3% (w/v).

13. A soluble pharmaceutical composition comprising pharmaceutically effective amounts of cyclic sodium hexametaphosphate or polymeric sodium metaphosphate, mannitol and glucosamine sulfate.

14. Composition according to claim 13 in which the amount of cyclic sodium hexametaphosphate or polymeric sodium metaphosphate is 0.5% (w/v).

15. Composition according to claim 13 in which the amount of mannitol is 3.17% (w/v).

16. Composition according to claim 13 in which the amount of glucosamine sulfate is 0.4% (w/v).

17. A soluble pharmaceutical composition comprising pharmaceutically effective amounts of cyclic sodium hexametaphosphate or polymeric sodium metaphosphate and glucosamine sulfate.

18. Composition according to claim 17 in which the amount of cyclic sodium hexametaphosphate or polymeric sodium metaphosphate is 0.75% (w/v).

19. Composition according to claim 17 in which the amount of glucosamine sulfate is 2.2% (w/v).

20. A pharmaceutical intra-articularly injectable formulation comprising a first container, containing the substance according to claims 1 to 3 in powder form, and a second container containing a solution of diluent in which at least one substance with anti-radical action and/or a substance with anti-oxidant action is dissolved, and wherein the substance of the first container is dissolved before use.

21. An injectable pharmaceutical formulation to be used for continuous washing of an articulation comprising a first container, containing the substance according to claims 1 to 3 in powder form, and a second container containing a solution of diluent in which at least one substance with anti-radical action and/or a substance with anti-oxidant action is dissolved, and in which the composition of the first container is dissolved before use.

22. A pharmaceutical containment formulation to be used after the solubilization of CPPD or HAP crystals in an articulation comprising a container containing a solution of diluent intra-articularly injectable,) slightly hypotonic, in which is dissolved at least one substance with anti-radical action of oxygen and anti-hypochlorite anion.

23. Aqueous hypotonic solution in which the substance according to claims 1 to 6 is dissolved.
